(19) United States
(12) Patent Application Publication
(10) Pub. No.: US 2011/0284005 A1
(43) Pub. Date: Nov. 24, 2011

(54) VALVE AND METHOD FOR FLOW CONTROL

(75) Inventor: Göran Cewers, Limhamn (SE)

(73) Assignee: MINDRAY MEDICAL SWEDEN AB, Sundbyberg (SE)

(21) Appl. No.: 13/105,649

(22) Filed: May 11, 2011

Related U.S. Application Data
(60) Provisional application No. 61/345,623, filed on May 18, 2010.

(51) Int. Cl. A61M 16/20

(52) U.S. Cl. 128/205.24

(57) ABSTRACT

The disclosure relates to a device for controlling a gas flow, e.g., from a breathing system connected to a patient at exhalation. The flow control is conducted by means of a flexible conduit with flexible circular segments being compressed along the length of the conduit, whereupon the flexible elements are collapsed towards a circular wall. The device comprises autoclavable parts and/or disposable parts, which can be separated from the breathing system without exposing staff handling the system to contaminated surfaces in the breathing system when changing patients.

### CROSS-REFERENCE TO RELATED APPLICATION(S)

This application claims the benefit of U.S. Provisional Application No. 61/345,623, filed May 18, 2010, which is incorporated herein by reference in its entirety.

### TECHNICAL FIELD

The following disclosure pertains to a valve. More particularly, the following disclosure relates to a valve for controlling a gas flow, such as a valve for controlling a gas flow in a breathing apparatus connected to a patient.

### SUMMARY OF THE INVENTION

A valve for medical ventilators may include a first flexible element having at least one flexible zone that is an articulation. The valve may also include at least one second flexible element having at least one flexible zone that is an articulation. The valve may further include a valve seat located either outside or inside of said first flexible element, as well as a channel between said first flexible element and said valve seat for a fluid to pass. The valve may also include at least one actuator unit arranged to axially compress or decompress said first flexible element for control of a flow of said fluid through said channel, wherein said first flexible element by a motion of said actuator unit is either angled radially towards or straightened up from said valve seat, and wherein at least a second flexible element is positioned to allow said axial movement.

A method of controlling a flow through at least one fluid passage may include axially compressing or decompressing at least two flexible elements of a valve, wherein said at least one first flexible element is angled radially towards or straightening up from a corresponding valve seat whereby said flow is controlled, wherein said at least one second flexible element is positioned to allow the relative axial movement occurring by said second flexible element to be either straightened up from or angled away from said fluid passage.

### BRIEF DESCRIPTION OF THE DRAWINGS

FIG. 1 is a schematic cross sectional view showing an axial cross section of a flexible conduit segment;

FIG. 2 is a schematic cross sectional view showing an axial cross section along a valve with the axis of a flexible conduit segment;

FIG. 3 is a schematic view showing an example of a valve configuration;

FIGS. 4-9 are schematic views of various examples of low pressure valves;

FIG. 10 is a schematic cross sectional view of an embodiment of a low pressure valve with an integrated flow meter being ultrasound transceiver elements; and

FIG. 11 is a schematic view showing how grooves in the flexible segment have been added to reduce the segment's axial compression resistance.

### DETAILED DESCRIPTION

When designing expiration valves for medical ventilators, the flow channel in the expiration valve should have low flow resistance and no turbulence, contaminated parts of the valve should be easy to clean, the valve should be small and light, and the actuator controlling the valve should be small and isolated from the flow channel.

Today, the most common design for expiration valves includes a circular disk lying against the end of a tube forming a valve seat, as illustrated in U.S. Pat. No. 5,127,400. The drawbacks of such a design are cleaning issues and the complexity of the flow channel, which causes turbulence. Moreover, the entire circular disk is pressurized, while the flow only depends on the outer edge of the disk. Thus, an unnecessarily strong, heavy, and expensive actuator is needed to control this type of valve.

To overcome these problems, a valve may include a first flexible element having at least one flexible zone and a valve seat, which is located either outside or inside of the flexible element in such a way that a fluid can pass a channel between the first flexible element and the valve seat. The valve may also include at least one actuator unit arranged to axially compress or decompress the first flexible element. In one embodiment, the first flexible element is radially angled towards or is straightened up from the valve seat, such that the flow of the fluid through the valve be controlled.

When the flexible element is in its normal position, the valve is open, and a flow of the fluid, which may be described as substantially without turbulence, can occur in the channel between the flexible element and the valve seat. A flexible element of the present disclosure may include one or more flexible zones that are operable as articulations in order for the flexible element to be controllably angled or protruded radially towards the valve seat when the flexible element is exposed to an axial compressive or decompressive motion from an actuator unit. With increasing angulation of the flexible element, at least one area of the flexible element will touch the valve seat. In one embodiment, the touching area is the flexible zone, and the area or zone may be designed so that a tight sealing effect occurs between the angled flexible element and the valve seat.

In one embodiment, the valve is rotationally symmetric with a coaxial arrangement of the valve seat and the flexible element.

The term “axial directed movement/motion” herein refers to a direction along or against the direction of flow, which takes place between two openings positioned at each side of the flexible element. The term “radial directed movement/motion” refers to movement substantially vertical to the direction of flow.

In some embodiments, the flexible elements of the valve are configured and positioned so they can be in at least one position where a side occurs having even characteristic on the flow side of the channel.

This disclosed design allows for an essentially non-turbulent flow in the open position, which decreases the flow resistance of the fluid through the flow channel.

In some embodiments, the valve includes at least a second flexible element, whereby the first flexible element is arranged to choke the flow, while at least one other flexible element is positioned so as to allow relative axial movement. As a result, deformation and wear of the first flexible element is avoided during repeated folding.

To facilitate the positioning of the valve between two non-flexible inlet and outlet channels, additional flexible elements may be utilized so that the axial compressive or decompressive movement of the valve can be conducted. This entails that strain is avoided on the material or on the mountings of the non-flexible inlet and outlet channels.

In some embodiments, the valve seat is provided as a rotationally symmetric circular wall placed at the centre of the first flexible element, such as the outside of a rod, or the inside or outside of a conduit.

In some embodiments, the valve seat has a rotationally symmetric, conic profile and is positioned at the centre of the first flexible element downstream. This design and location of the valve seat facilitates the creation of an essentially non-turbulent flow.

Valves may also be designed where the valve seat is a rotationally symmetric circular wall, which may include the inside of a conduit placed around the first flexible element.

In another embodiment, the construction material in the flexible element of the valve and the valve seat is autoclavable and/or the construction material in the flexible element and the valve seat is disposable, or the design may comprise parts made of autoclavable material combined with parts that are disposable. Examples of such materials include silicone rubber, stainless steel, etc.

Choosing these materials allows the valve to be used for medical devices, such as breathing apparatuses. Such a valve might be an expiration valve in a respirator. Thus, valves contaminated by patients may thereby be safely cleaned and disinfected between patients.

In some embodiments, the actuator unit of the valve is at least one piezoelectric actuator. In other embodiments, the actuator unit may include at least one coil actuator. A skilled artisan will recognize that still other types of actuator units may be suitable.

In one embodiment, the valve actuator unit may be arranged without contact with the flow channel, thus the actuator unit need not be autoclavable. This simplifies handling and increases the useful life of the actuator unit.

In some embodiments, the valve has an integrated flow meter. In particular, at least two ultrasound transceivers may be placed along the flow channel to measure the flow through the channel, allowing a compact unit to be provided. The unit may allow rapid control of the flow through the valve, since the distance between the flow meter and the valve may be kept short, and turbulence may be thus avoided.

In another aspect, the disclosure includes a method for controlling a flow through at least one fluid passage, where the method comprises axially compressing or decompressing at least one flexible element of a valve, wherein the at least one flexible element is angled radially towards or straightened up from a corresponding valve seat. The valve may be designed as described above.

The method provides for a substantially non-turbulent flow of one or more fluids through a flow channel, and the flow may be easily choked when needed. The flow control is rapid and reliable with a compact unit. Furthermore, piezo actuators may be used providing low energy consumption.

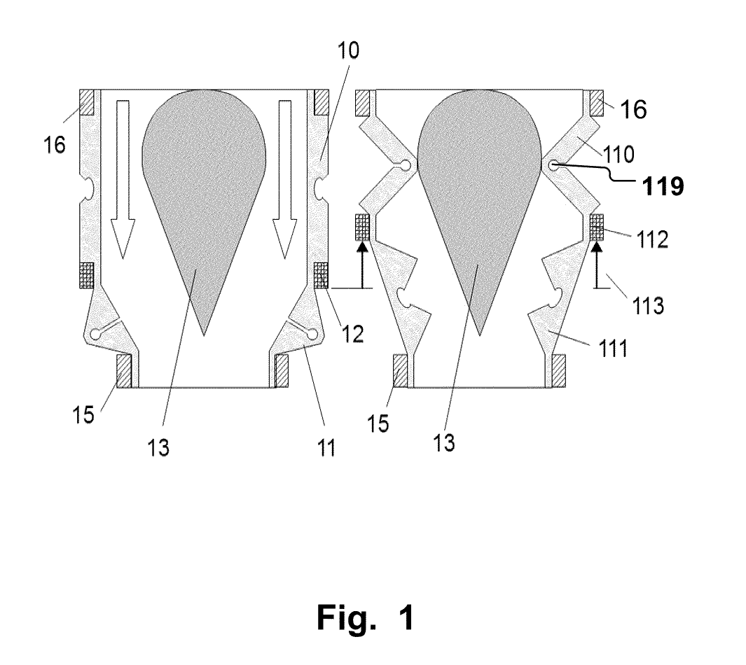

A device, according to the present disclosure, may be obtained using a soft conduit fixed at the ends, manufactured of, e.g., silicone rubber, with flexible segments 10, 11, as shown in FIG. 1. A movable ring exposes the flexible conduit to an axial movement between the ends so that the flexible element is affected.

FIG. 1 is a schematic view showing an axial cross section of a flexible conduit segment. FIG. 1 shows one and the same valve arrangement in two different states. The left hand drawing shows the valve device in an open position, and the right hand drawing shows the valve device in a closed position.

The left hand drawing shows a first flexible element 10 in an uncompressed state, while a second flexible element 11 is in a compressed state. The circular protrusion 119 of the first flexible element 10 acts as a soft valve element. In this state, the valve device is normally open.

In one embodiment, a rotationally symmetric hard body 13 is centered inside the flexible conduit, which has been aerodynamically designed to minimize the flow resistance in the valve device. The body 13 is a valve seat towards which the first flexible element 10 operates. Body 13 is fastened to fastening rings 15, 16 by supporting elements, which are not shown in the figures. Fastening rings 15, 16 are arranged at each end of the flexible element, which comprises the first and second flexible elements 10, 11 as an integrated part.

When the valve device is open, as shown in the left hand drawing of FIG. 1, the inside of the flexible conduit is virtually even. This, combined with the design of the centre body 13, causes very low flow resistance in the valve device.

In one embodiment, the valve device is closed by axially moving ring 12 the distance 113 towards the fastening ring 16, so that the flexible conduit segment 110 is pressed against the body 13. The conduit segment 111 will be stretched at the same time. In this position, the flow profile is no longer at optimum, but this is relatively unimportant, as there is no flow through the valve device in the closed position. Ring 112 may also be positioned between a closed and an open position. In this case, the valve device acts as a proportional valve. Conduit segment 110 has a profile that differs slightly compared to the conduit segment 111, in that the triangular shaped sections are stripped at the top to reduce the weight of the segment, thus raising the system's resonance frequency. The segment 111 may also be made lighter in the same way. The ring 12 may also be made extra light, e.g., by forming its cross section in a U-shape, T-shape, or the like.

Unlike conduit segment 110, the inner profile of the conduit segment 111 may be conically shaped when the valve device is open. As a result, the flow profile is more favourable while less movement needs to be absorbed by this segment, thereby it can be made smaller and lighter, which helps to increase the resonance frequency in the system and improves regulating properties.

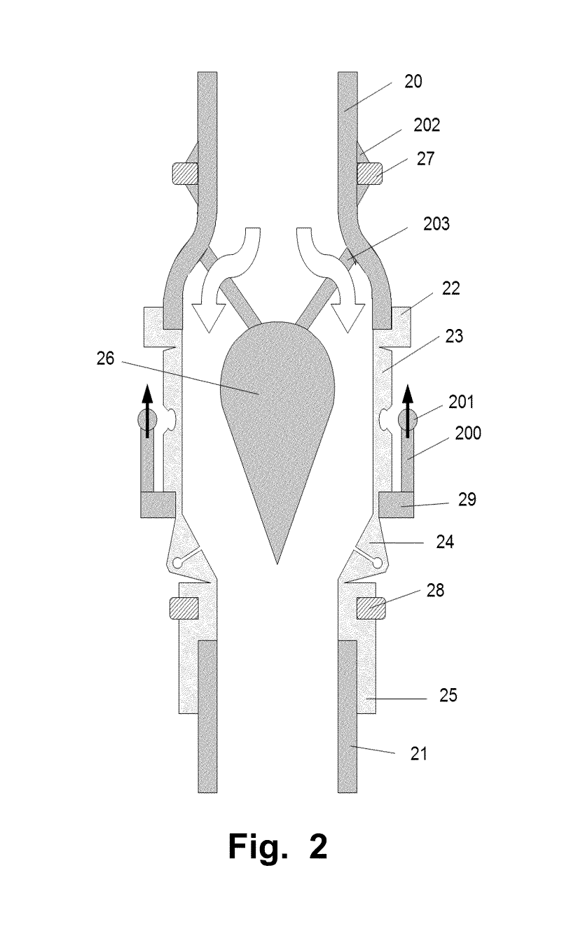

FIG. 2 is a schematic view showing an exemplary embodiment in axial cross section along the axis of a flexible conduit segment. One embodiment of the disclosure includes a device, as illustrated in FIG. 2, which can be dismounted from the chassis of the device it is intended to be placed in, such as a respirator, without having to open the patient's exhalation tube system, such that the chassis is left uncontaminated. The tube system can thereafter be moved for cleaning, destruction, or recycling.

When the device shown in FIG. 2 is dismounted, only parts 27, 28, 200 and 201 remain in the chassis member. These parts belong to a part of the device, i.e., the actuator portion. The other parts belong to the valve portion.

After the valve portion has been dismounted, the patient tubes can be removed from end parts 20 and 21. The valve portion of the device includes three parts which can be separated and autoclaved.

The first part of the valve part comprises a first end part of a hard material, such as plastic. This part forms the inlet of the expiration valve and includes end part 20, which is also an inlet, fastening means 202, and supporting elements 203, which hold central body 26.

The second part of the valve portion may include a soft conduit made of, e.g., silicone rubber, with two flexible sections 23 and 24, as well as end adaptors 22 and 25. A guide ring 29 of a hard material, such as plastic, is mounted over the soft conduit of the valve portion, as shown. The purpose of ring 29 is to transfer movement from the actuator portion to an axial compression of the first flexible segment 23 to force it radially against the central body 26 when the valve is to be closed.

The third part of the valve portion may include a second end part of a hard material, such as plastic. This part forms the outlet for the expiration valve and comprises the end part 21.

The actuator portion comprises a flexible foil 200, which upon application of the valve portion in the chassis, hooks onto the guide ring 29 and a supporting element 201, which, when the valve is closed, is moved in the direction of the arrow, as shown in FIG. 2.

Supporting element 201 is then connected to an actuator, which may be electromagnetic, thermal, chemical, magnetostrictive, or piezoelectric.

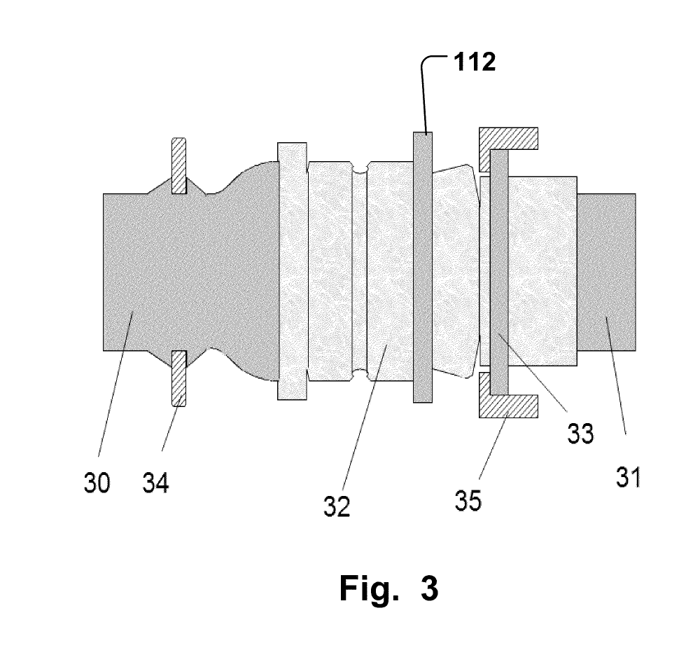

FIG. 3 is a schematic view showing one exemplary embodiment of a valve configuration. The valve portion in FIG. 3 is viewed from above. Inlet part 30, the soft sectioned conduit 32 with guide ring 112, fastening ring 33, and outlet part 31 are shown. The holders 34 and 35 are anchored to the chassis. The ring 112 is used to close and/or open the valve. The opening of the valve can be conducted by restoring the elasticity to the conduit 32.

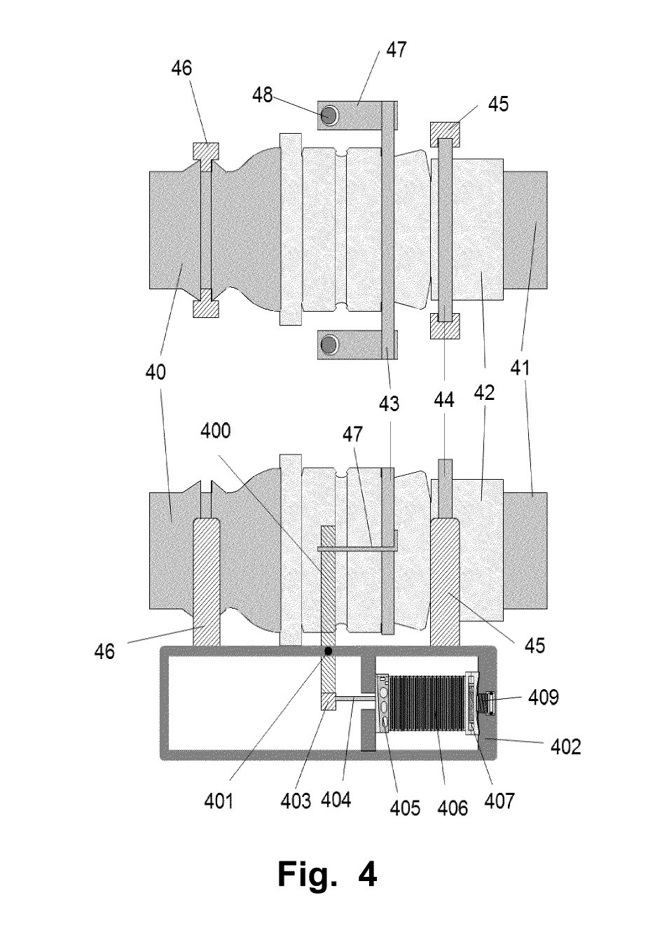

FIG. 4 is an example showing how an actuator may be connected to the valve portion. FIG. 4 shows how a lever 48, which may be, e.g., U or Y shaped, transmits the movement from actuator 406 via a mechanical motion amplifier 405, axis 404, and lower part of lever 403 to the mobile circular ring 43 via an articulation 400, controlled via the pivoting point 401, such as a hinge or a flexible pivot and an articulation 47. The friction is thereby kept low. The actuator 406 may be finely adjusted using adjustment device 409. A temperature compensating unit 407 may also be included. Holders or guiding means 45 and 46 are fastened to the chassis to snap onto the valve body. A fastening ring 44 holds the soft rubber in place and comes with the valve unit when it is lifted. Inlet 40 and outlet 41 are connections to the valve unit, e.g., for 22 mm conduits. FIG. 4 also illustrates end adapter 42, which is similar to the end adapter 25 of FIG. 2, and a housing 402.

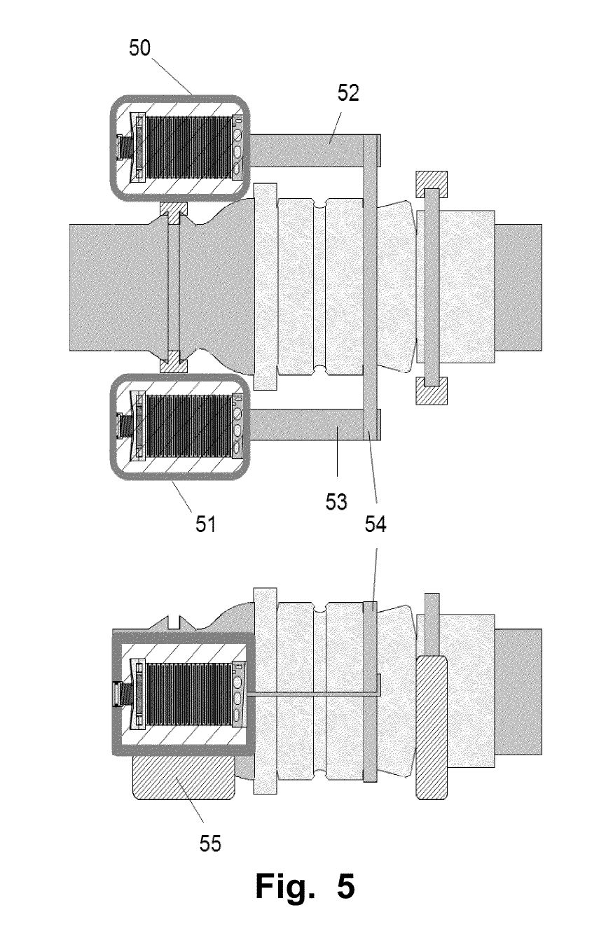

FIG. 5 is a schematic view showing an exemplary embodiment of a low pressure valve with a flexible conduit surrounding a rotationally symmetric body. The upper part of FIG. 5 is a view from above, while the bottom part of FIG. 5 is a side view. In this variant of the valve device, the outward movement towards the flexible conduit is controlled using two actuators. There are two piezoelectric actuators 50 and 51 with flexible linking elements 52 and 53 anchored to the chassis of the valve device. The valve device can be removed from the parts belonging to the chassis and then, for example, be autoclaved. A bearing element 55 is anchored to the chassis. FIG. 5 also illustrates circular ring 54, which is similar to the circular ring 43 of FIG. 4.

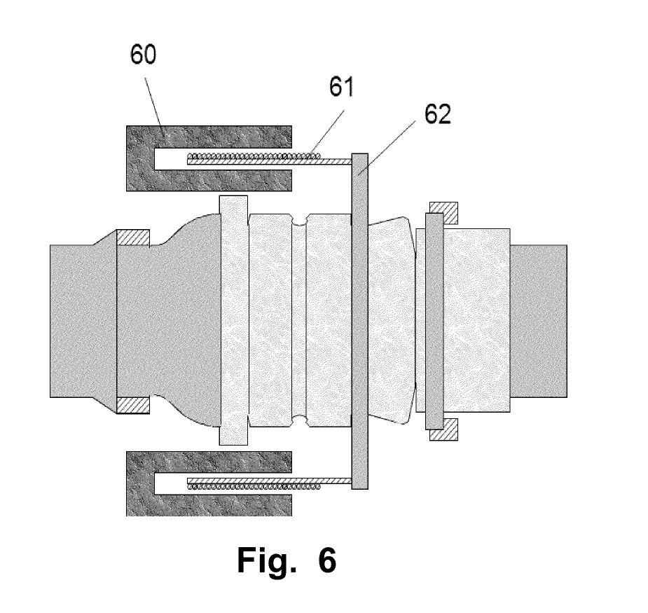

FIG. 6 is a schematic view showing an exemplary embodiment of a version of a low pressure valve. Instead of piezo actuators, as in FIG. 5, the valve device can be controlled by an electromagnetic coil actuator 60, as shown in FIG. 6. Here the coil 61 acts directly against the movable disk 62.

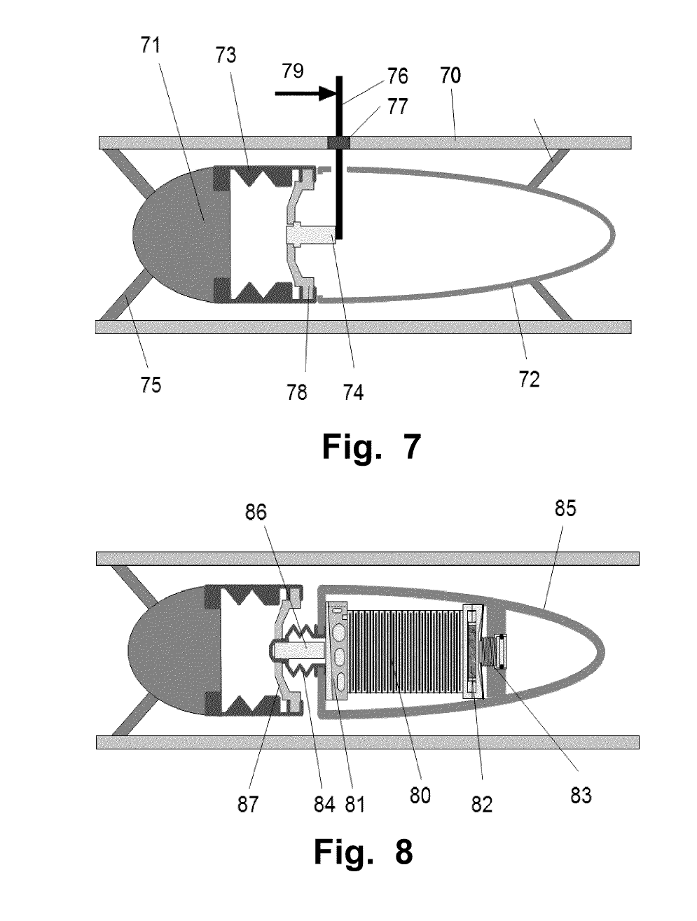

FIG. 7 is a schematic view showing an exemplary embodiment of a low pressure valve with a hard conduit 70 surrounding a flexible conduit 71. Disk 78 is moved by a lever 76 along the distance 79 when the valve device is brought to a closed position. As an alternative to a conduit surrounding a body, the valve device may be made with a hard surrounding conduit and a flexible inner conduit, as shown in FIG. 7. Here, a movement 79 is transmitted by a lever 76 using an articulation and seal 77 to the axis 74 and further on to the movable disk 78, which deforms the movable segment 73 out towards the inside of body 70. FIG. 7 also illustrates a body 72, as well as a support element 75.

FIG. 8 is a schematic view showing an exemplary embodiment of a low pressure valve with the same geometry as in FIG. 7, but where the lever has been replaced by a piezo actuator 80, which is encapsulated in the actual flow channel. The ring 87 is moved using the mechanical amplifier 81 when the valve device is brought to the closed position. The bellows 84 isolates the environment of the actuator 80 from the gas channel. FIG. 8 also illustrates a temperature compensation means 82, a trimming device 83, a body 85, and a peg/rod 86 between the actuator unit and the choking part of the valve.

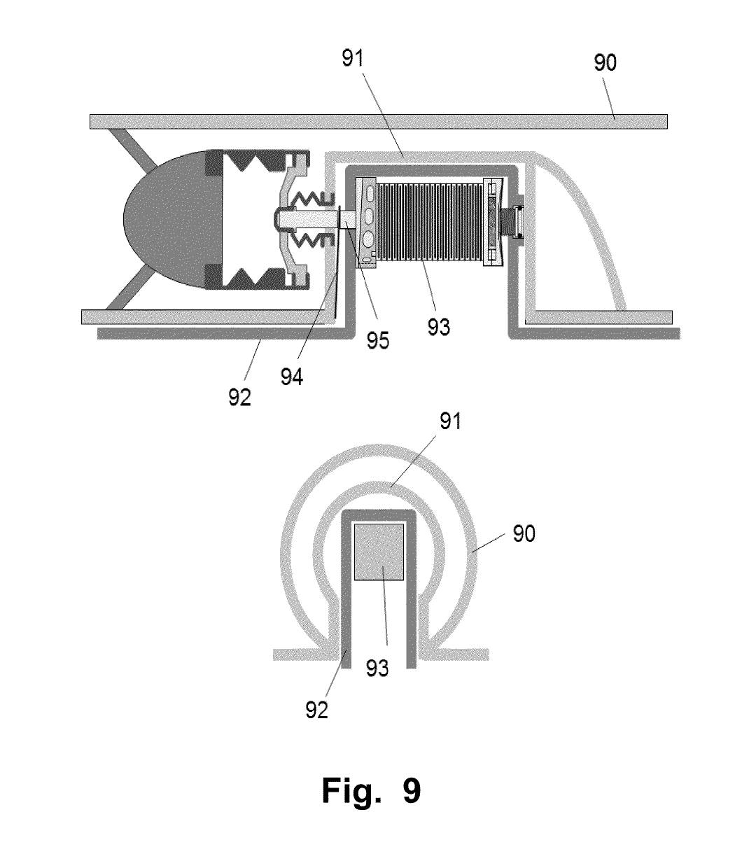

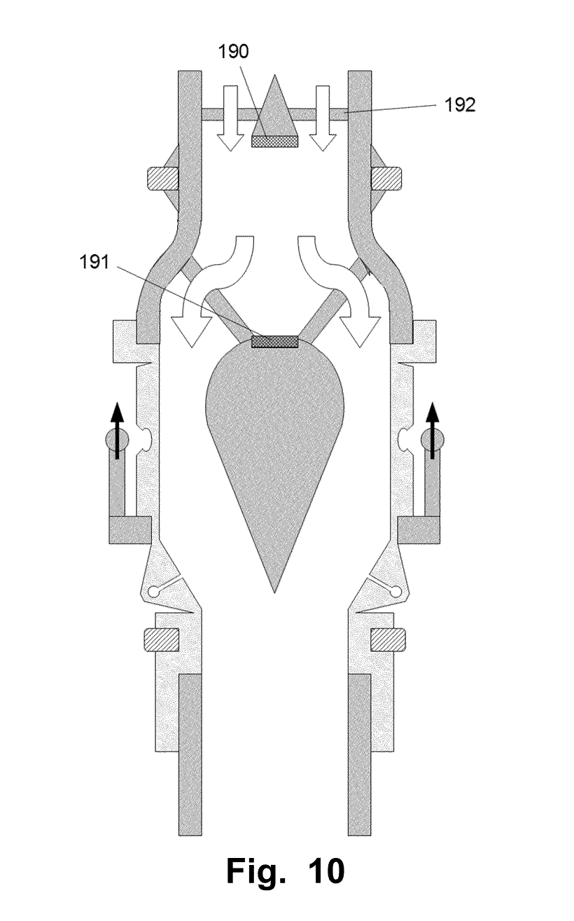

FIG. 9 shows a schematic view of an exemplary embodiment of a low pressure valve with geometry similar to that of FIG. 8, but where a cavity 91 has been made in the housing 90. This cavity encloses a housing 92, which is anchored to the chassis of the ventilator. The actuator element 93 is located in this space. In this design, the device enclosed in conduit 90 may be removed from the actuator portion. A resilient element 94, combined with a supporting element 95, ensures that the actuator movement is transmitted to the valve device and that the units may be docked.

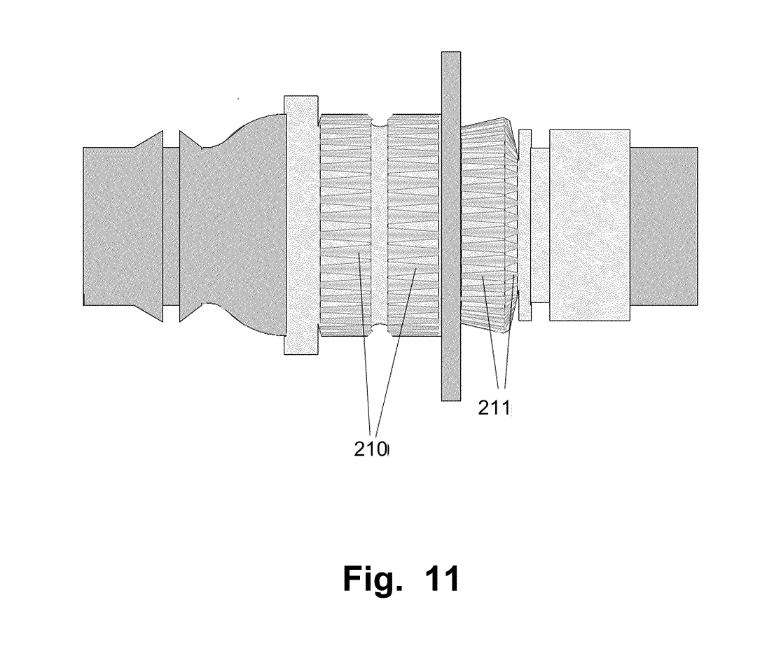

FIGS. 10 and 11 are schematic views showing an exemplary embodiment of a low pressure valve with the same basic design as in FIG. 6, but ultrasound transceiver elements 190 and 191 have been added. The ultrasound transceiver element 190 is fixed in the valve device inlet by the supporting elements 192.

FIG. 11 is a schematic view showing an exemplary embodiment where substantially longitudinal grooves 110 and 111 on the outside of the flexible segments have been added to decrease the axial compression resistance of the segments.

Without further elaboration, it is believed that one skilled in the art can use the preceding description to utilize the present disclosure to its fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles of the disclosure described herein. In other words, various modifications and improvements of the embodiments specifically disclosed in the description above are within the scope of the appended claims. The scope of the invention is, therefore, defined by the following claims. The words “including” and “having,” as used herein, including the claims, shall have the same meaning as the word “comprising.”

What is claimed is:

1. A valve for medical ventilators comprising:
a first flexible element having at least one flexible zone that is an articulation;
at least one second flexible element having at least one flexible zone that is an articulation;
a valve seat located either outside or inside of said first flexible element;
a channel between said first flexible element and said valve seat for a fluid to pass; and
at least one actuator unit arranged to axially compress or decompress said first flexible element for control of a flow of said fluid through said channel, wherein said first flexible element by a motion of said actuator unit is either angled radially towards or straightened up from said valve seat, and wherein at least a second flexible element is positioned to allow said axial movement.

2. The valve according to claim 1, wherein said at least one flexible zone is operable as an articulation arranged to angle said flexible element radially.

3. The valve according to claim 1, wherein said first flexible element has a touching area configured to, after said first flexible element is angled radially a relative distance, touch said valve seat with said touching area.

4. The valve according to claim 3, wherein said touching area includes said flexible zone.

5. The valve according to claim 1, wherein said first flexible element has a zone configured so that a sealing effect occurs when it touches said valve seat.

6. The valve according to claim 1, wherein said flexible elements and said valve seat are arranged and positioned relative to each other so that said flexible elements are arranged in at least one position that allows a flow substantially without turbulence through said channel of said valve.

7. The valve according to claim 1, wherein said flexible element is configured to, in at least one position, have a side with even characteristic located on a flow side of said channel.

8. The valve according to claim 1, wherein said first and second flexible elements are an integrated part.

9. The valve according to claim 1, wherein said axial motion is along or against the direction of said flow.

10. The valve according to claim 1, wherein said radial motion occurs substantially vertically relative to the direction of said flow.

11. The valve according to claim 1, wherein said valve seat is a rotationally symmetric circular wall located in the center of said first flexible element.

12. The valve according to claim 1, wherein said valve seat has a rotationally symmetric conical profile and is located in the center of said first flexible element downstream.

13. The valve according to claim 1, wherein said valve seat is a rotationally symmetric circular wall that is the inside of a conduit positioned around said first flexible element.

14. The valve according to claim 1, wherein at least a portion of construction material of said first flexible element and said valve seat is autoclavable.

15. The valve according to claim 1, wherein at least a portion of construction material of said flexible element and said valve seat is disposable.

16. The valve according to claim 1, wherein said actuator unit is at least one piezoelectric actuator.

17. The valve according to claim 1, wherein said actuator unit is arranged without contact to the channel.

18. The valve according to claim 1, wherein at least two ultrasound transceivers are located along the channel for measuring said flow through said channel.

19. The valve according to claim 1, wherein substantially longitudinal grooves are arranged within the outside of said flexible elements.

20. A method of controlling a flow through at least one fluid passage, wherein the method comprises axially compressing or decompressing at least two flexible elements of a valve, wherein said at least one first flexible element is angled radially towards or straightening up from a corresponding valve seat whereby said flow is controlled, wherein said at least one second flexible element is positioned to allow the relative axial movement occurring by said second flexible element to be either straightened up from or angled away from said fluid passage.
